(19) United States
(12) Patent Application Publication
(10) Pub. No.: US 2016/0296498 A1
(43) Pub. Date: Oct. 13, 2016

(54) Ketorolac Sublingual Spray Formulations

(71) Applicant: Insys Development Company, Inc., Chandler, AZ (US)

(72) Inventors: Kiran Amancha, Chandler, AZ (US); Horng-Shin Li, Chandler, AZ (US); Ningxin Yan, Chandler, AZ (US); Venkat Goskonda, Phoenix, AZ (US)

(21) Appl. No.: 15/075,438

(22) Filed: Mar. 21, 2016

Related U.S. Application Data
(60) Provisional application No. 62/145,038, filed on Apr. 9, 2015.

(51) Int. Cl. A61K 31/407; A61K 47/12; A61K 47/22; A61K 9/00

(52) U.S. Cl. CPC A61K 31/407; A61K 9/006; A61K 47/12; A61K 47/22

(57) ABSTRACT

The invention is directed to room temperature storage stable sublingual spray formulations containing ketorolac. The invention is further directed to methods of treating pain by administering sublingual spray formulations containing ketorolac to patients in need of such treatments.

### FIELD OF THE INVENTION

The invention is directed to sublingual spray formulations containing ketorolac. The invention is further directed to methods for treating pain by administering sublingual spray formulations containing ketorolac to patients in need of pain relief.

### BACKGROUND OF THE INVENTION

Ketorolac is a non-steroid anti-inflammatory drug with the following structure:

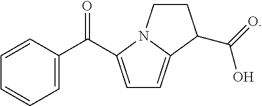 [SMILES: O=C(c1ccccc1)c1ccc2n1CCC2C(=O)O]

Ketorolac is commercially available as a tablet, capsule or injection to treat moderate to severe pain. For example, ketorolac has been used to treat postoperative pain and pain from migraine headaches.

Tablets and capsules must be taken orally by the patient. Oral routes of administration are not desirable as they can lead to negative side effects such as vomiting, have slow absorption rates, and poor overall absorption rates.

Further, ketorolac is rapidly metabolized by the body and requires frequent administration. When ketorolac is administered via injection the patient must repeatedly tolerate skin injections or have an intravenous drip. These methods are not convenient and increase the risk of infection.

U.S. Pat. No. 7,879,901 is directed to sublingual ketorolac tablets containing 30 to 50% lactose, 3 to 9% sorbitol, and 9 to 17% cellulose. The tablets must be dissolved in the mouth by the patient which is a skill that some potential patients, such as small children, may not possess.

“Sublingual” means “under the tongue” and refers to administration of a substance via the mouth in such a way that the substance is rapidly absorbed via the blood vessels under the tongue. A sublingual formulation is desirable because it bypasses hepatic first pass metabolic processes which provide better bioavailability, rapid onset of action, and higher patient compliance. Dysphagia (difficulty in swallowing) is common among in all ages of people and more common in geriatric patients. In terms of permeability, the sublingual area of oral cavity is more permeable than buccal area. Sublingual drug administration is applied in field of cardiovascular drugs, analgesics, steroids, enzymes and barbiturates.

A challenge of creating sublingual spray formulations is that it must be capable of producing spray droplets that are over 10 microns in diameter. Spray droplets 10 microns or smaller could be inhaled into the lungs. The optimal particle size for sublingual spray droplets is from 20 to about 200 microns in diameter. It is desirable for the formulation to create droplet sizes near 20 because this increases the surface area and increased surface area exposure is one factor that contributes to a high bioavailability. Sublingual formulations should be able to maintain a consistent droplet size throughout its shelf life.

U.S. Pat. No. 6,720,001 is directed to pharmaceutical emulsions containing an aqueous phase, an emulsifier, and a polar oil phase containing one or more structured triglycerides. These oil-in-water emulsions can contain ketorolac. However, this patent fails to provide storage stable ketorolac formulations.

US Patent Application Publication No. 2009/0246273 is directed to sublingual ketorolac formulations that require ethanol and propylene glycol. However, this application fails to provide ketorolac formulations that are storage stable and capable of producing excellent droplet size distributions during administration.

Accordingly, while there are various ketorolac formulations currently available, there is still a need in the art for an aqueous quick-onset, storage stable sublingual spray formulation containing ketorolac.

### SUMMARY OF THE INVENTION

In one aspect, the present invention is directed to room temperature storage stable sublingual spray formulations comprising from about 0.1% w/w to about 35% w/w ketorolac or a salt thereof, from about 0.1% w/w to about 95% w/w water, from about 0.001% w/w to about 1% w/w of a buffer salt, and from about 0.001% w/w to about 1% w/w antioxidant, wherein the pH of the formulation is from about 5 to about 9.

In another aspect, the present invention is directed to methods for alleviating pain comprising administering the formulations of the present invention to a patient.

### DETAILED DESCRIPTION

Applicant unexpectedly discovered sublingual ketorolac formulations that have improved bioavailability, a more rapid onset of action, and improved storage stability (see, for example, Example 2 below), and are capable of producing excellent droplet size distribution profiles (see, for example, Example 3 below). Further, Applicant found that the buffer salt is critical in the formulation as it creates an optimal pH range of from about 5 to about 9. Applicant found that ketorolac would degrade when stored at room temperature if formulated with a pH of less than 5 or greater than 9.

In an embodiment, the present invention is directed to room temperature storage stable sublingual spray formulations comprising from about 0.1% w/w to about 35% w/w ketorolac or a salt thereof, from about 0.1% w/w to about 95% w/w water, from about 0.001% w/w to about 1% w/w of a buffer salt, and from about 0.001% w/w to about 1% w/w antioxidant, wherein the pH of the formulation is from about 5 to about 9.

In a preferred embodiment, the ketorolac salt is ketorolac tromethamine. Other pharmaceutically acceptable salts may be used. For example, suitable salts could include citrate, hydrochloride, halide, sulfate, phosphate, acetate, maleate, succinate, tromethamine, ascorbate, carbonate, mesylate and lactate.

In another embodiment, the formulations of the present invention comprise about 5% w/w to about 35% w/w ketorolac or a salt thereof. In a preferred embodiment, the formulations contain from about 10% w/w to about 35% w/w ketorolac or a salt thereof. In one more preferred embodiment, the formulations contain from about 10% w/w to about 20% w/w, or from about 13% w/w to about 17% w/w ketorolac or a salt thereof. In another more preferred embodiment, the formulations contain from about 25% w/w to about 35% w/w, or from about 29% w/w to about 33% w/w ketorolac or a salt thereof.

In a preferred embodiment, the pH is from about 6 to about 8.

In an embodiment, the formulations contain a buffer salt that is selected from the group consisting of a sodium, potassium, or calcium salt of citric acid, acetic acid, phosphoric acid, boric acid malic acid, adipic acid, fumaric acid, tartaric acid, palmitic acid, and a combination thereof In a preferred embodiment, the buffer salt is sodium citrate. In a more preferred embodiment, the buffer salt is sodium citrate and the pH of the formulation is between 6 and 8.

In another embodiment, the formulations comprise from about 0.01% w/w to about 1% w/w of the buffer salt. In a preferred embodiment, the formulations comprise from about 0.05% w/w to about 0.8% w/w of the buffer salt.

In a preferred embodiment, the formulations comprise from about 1% w/w to about 95% w/w water. In a more preferred embodiment, the formulations comprise from about 50% w/w to about 95% w/w water. In a most preferred embodiment, the formulation comprise from about 65% w/w to about 85% w/w water.

In yet another embodiment, the formulations comprise a solvent selected from the group consisting of ethyl alcohol, propylene glycol, glycerol, polyethylene glycol, and a combination thereof.

In an embodiment, the formulations of the present invention can be propellant free. Preferably the formulations of the present invention are sublingual spray formulations.

In an embodiment, the formulations of the present invention comprise an antioxidant, permeation enhancer, sweetener, sweetness enhancer, flavoring agent, preservative, or a combination thereof.

Suitable antioxidants include, but are not limited to, ascorbyl palmitate, ascorbic acid, sodium ascorbate, alpha tocopherol, butylated hydroxytoluene, butylated hydroxyanisole, cysteine HCl, citric acid, ethylene diamine tetra acetic acid (EDTA), methionine, sodium metabisulfite, sodium bisulfate, propyl gallate, thioglycerol, and combinations thereof In a preferred embodiment, the antioxidant is sodium ascorbate.

In a further embodiment, the formulations comprise a permeation enhancer. Suitable permeation enhancers include, but are not limited to, oleic acid, polysorbate 80, menthol, EDTA, sodium edetate, cetylpyridinium chloride, sodium lauryl sulfate, citric acid, sodium desoxycholate, sodium deoxyglycolate, glyceryl oleate, L-lysine, and combinations thereof.

If the formulations contain a permeation enhancer, the formulations preferably contain from about 0.001% w/w to about 1% w/w permeation enhancer.

In another embodiment, the formulations comprise a sweetener. Suitable sweeteners include, but are not limited to, sucrose, aspartame, saccharin, dextrose, mannitol, xylitol, and combinations thereof.

If the formulations contain a sweetener, the formulations preferably contain from about 0.001% w/w to about 1% w/w sweetener.

In yet another embodiment, the formulations comprise a sweetness enhancer. Suitable sweetness enhancers include, but are not limited to, the ammonium salt forms of crude and refined Glycyrrhizic Acid. Magnasweet® products (available from Mafco Worldwide Corporation, Magnasweet is a registered trademark of Mafco Worldwide Corporation) use the ammonium salt forms of crude and refined Glycyrrhizic Acid. Glycyrrhizic Acid is also available as a pure derivative in the sodium and potassium salt forms.

If the formulations contain a sweetness enhancer, the formulations preferably contain from about 0.001% w/w to about 1% w/w sweetness enhancer.

In an embodiment, the formulations comprise a flavoring agent. Suitable flavoring agents include, but are not limited to, raspberry, peppermint oil, grape flavor, menthol, spearmint oil, citrus oil, cinnamon oil, strawberry flavor, cherry flavor, raspberry flavor, orange oil, lemon oil, lemon mint flavor, fruit punch flavor, and combinations thereof In a preferred embodiment, the formulations contain fruit punch flavor, raspberry flavor, grape flavor, or lemon mint flavor.

If the formulations contain a flavoring agent, the formulations preferably contain from about 0.001% w/w to about 1% w/w flavoring agent. In a more preferred embodiment, the formulations contain from about 0.005% w/w to about 0.5% w/w of the flavoring agent.

In an embodiment, the formulations comprise a preservative. Suitable preservatives include, but are not limited to, butyl paraben, methyl paraben, ethyl paraben, propyl paraben, sodium benzoate, benzoic acid, sorbic acid, and combinations thereof In a preferred embodiment, the preservatives are methyl paraben and propyl paraben.

If the formulations contain a preservative, the formulations preferably contain from about 0.001% w/w to about 1% w/w preservative. In a more preferred embodiment, the formulations contain from about 0.005% w/w to about 0.5% w/w of the preservative.

In yet another embodiment, the present inventions is directed to room temperature storage stable, sublingual spray formulation comprising from about 10% w/w to about 20% w/w ketorolac salt, from about 65% w/w to about 85% w/w water, from about 0.001% w/w to about 1% w/w of a buffer salt selected from the group consisting of a sodium salt of citric acid, phosphoric acid, and a combination thereof, from about 0.001% w/w to about 1% w/w of a buffer salt, and from about 0.001% w/w to about 1% w/w antioxidant, wherein the pH of the formulation is from about 5 to about 9. In an embodiment, this formulation is propellant free.

In an alternative embodiment, the present invention is directed to methods for treating pain comprising administering the formulations of the present invention to a patient in need pain relief.

In a preferred embodiment, the formulations of the present invention are administered with a spray pump. In a more preferred embodiment, the spray pumps deliver about 50 to 200 μL of the formulations of the present invention under the patient's tongue.

In a preferred embodiment, the formulations of the present invention provide pain relief caused by a migraine headache.

In another preferred embodiment, the formulations of the present invention provide pain relief wherein the pain is a result of a surgery.

In a preferred embodiment, the ketorolac in the formulations of the present does not degrade when stored at 40° C. or 55° C.

In yet another embodiment, the formulations of the present invention are capable of producing a droplet size distribution wherein the mean Dv(10) is from about 10 to about 170 microns during administration. Preferably, the formulations of the present invention are capable of producing a droplet size distribution wherein the mean Dv(10) is from about 18 to about 25 microns during administration.

In a further embodiment, the formulations of the present invention are capable of producing a droplet size distribution wherein the mean Dv(50) is from about 20 to about 315 microns during administration. Preferably, the formulations of the present invention are capable of producing a droplet size distribution wherein the mean Dv(50) is from about 25 to about 75 microns during administration.

In yet another embodiment, the formulations of the present invention are capable of producing a droplet size distribution wherein the mean Dv(90) is from about 60 to about 585 microns during administration. Preferably, the formulations of the present invention are capable of producing a droplet size distribution wherein the mean Dv(90) is from about 350 to about 470 microns during administration.

In an embodiment, the formulations of the present invention are capable of producing a spray span ((Dv90−Dv10)/Dv50) of from about 1 to about 10.

In yet another embodiment, the formulations of the present invention are capable of producing a Dmin of from about 20 to about 30 millimeters during administration.

In a further embodiment, the formulations of the present invention are capable of producing a Dmax of from about 20 to about 55 millimeters during administration. Preferably, the formulations of the present invention are capable of producing a Dmax of from about 20 to about 35 millimeters during administration.

In another embodiment, the formulations of the present invention are capable of producing an ovality ratio of from about 1 to about 2.5 during administration.

In yet another embodiment, the formulations of the present invention are capable of producing a plume width of from about 15 to about 45 millimeters during administration. Preferably, the formulations of the present invention are capable of producing a plume width of from about 20 to about 30 millimeters during administration.

In another embodiment, the formulations of the present invention are capable of producing a spray plume angle of from about 30 to about 60 degrees during administration. Preferably, the formulations of the present invention are capable of producing a spray plume angle of from about 35 to about 50 degrees during administration.

As used herein, “ketorolac” refers to the base or a pharmaceutically acceptable salt, ester, derivative, or prodrug thereof.

As used herein, “propellant free” refers to a formulation that is not administered using compressed gas.

As used herein, “room temperature storage stable” refers to formulations which maintain greater than 95% purity following twelve weeks of storage at about 40° C.

As used herein, all numerical values relating to amounts, weights, and the like, that are defined as “about” each particular value is plus or minus 10%. For example, the phrase “about 10% w/w” is to be understood as “9% w/w to 11% w/w.” Therefore, amounts within 10% of the claimed value are encompassed by the scope of the claims.

As used herein “% w/w” and “percent w/w” refer to the percent weight of the total formulation.

As used herein the term “effective amount” refers to the amount necessary to treat a patient in need thereof.

As used herein the term “patient” refers, but is not limited to, a person that is being treated for pain.

As used herein the term “pharmaceutically acceptable” refers to ingredients that are not biologically or otherwise undesirable in a sublingual dosage form.

The disclosed embodiments are simply exemplary embodiments of the inventive concepts disclosed herein and should not be considered as limiting, unless the claims expressly state otherwise.

The following examples are intended to illustrate the present invention and to teach one of ordinary skill in the art how to use the formulations of the invention. They are not intended to be limiting in any way.

### EXAMPLES

### Example 1

### Preparation of Ketorolac Sublingual Formulations

In order to prepare ketorolac sublingual formulations, the components as indicated in “Table 1. The Components of Formulations 1A to 1J” below were weighed. The components were mixed until a clear solution was formed. Ketorolac tromethamine was used as the source of ketorolac salt in the formulations. Each formulation had a pH between 6 and 8.

TABLE 1
The Components of Formulations 1A to 1J

| | 1A | 1B | 1C | 1D | 1E | 1F | 1G | 1H | 1I | 1J |
|---|---|---|---|---|---|---|---|---|---|---|
| | % w/w | % w/w | % w/w | % w/w | % w/w | % w/w | % w/w | % w/w | % w/w | % w/w |
| Ketorolac salt | 15.75 | 15.75 | 15.75 | 15.75 | 15.2 | 15.2 | 15.2 | 15.75 | 31.5 | 15.2 |
| Sodium citrate | 0.5 | 0.5 | 0.5 | 0.5 | 0.5 | 0.5 | 0.5 | — | 0.5 | 0.5 |
| Sodium ascorbate | 0.02 | 0.02 | 0.02 | 0.02 | 0.02 | 0.02 | 0.02 | — | 0.02 | 0.02 |
| Sodium Phosphate | — | — | — | — | — | — | — | 0.95 | — | — |
| Dibasic Anhydrous | | | | | | | | | | |
| Ascorbic acid | — | — | — | — | — | — | — | 0.25 | — | — |
| Flavoring | — | 0.15 | — | — | 0.1 | 0.1 | 0.08 | — | — | — |
| Methyl paraben | — | — | 0.02 | 0.02 | 0.02 | 0.02 | 0.02 | — | — | 0.02 |
| Propyl paraben | — | — | — | 0.02 | 0.02 | 0.02 | 0.02 | — | — | 0.02 |
| Sodium Thiosulfate | — | — | — | — | — | — | — | 0.015 | — | — |
| Propylene glycol | — | — | — | — | — | — | — | 10 | — | — |
| Polyvinylpyrrolidone | — | — | — | — | — | — | — | 5 | — | — |
| Water | 83.7 | 83.6 | 83.7 | 83.7 | 84.1 | 84.1 | 84.2 | 68.0 | 68.0 | 84.2 |

### Example 2

### Stability of Ketorolac Sublingual Formulations

In order to determine the stability of formulations of the present invention, several formulations were subjected to standard stability testing. Specifically, the formulations were stored at 40° C.±2° C./75%±5% relative humidity and at 55° C. The results of these tests are below in Tables 2 to 8.

TABLE 2
Stability of Formulation 1A

| | | | 40° C. | | | | 55° C. | | | | |
|---|---|---|---|---|---|---|---|---|---|---|---|
| | RRT | Limit | Time 0 | 2 wk. | 4 wk. | 8 wk. | Time 0 | 2 wk. | 4 wk. | 6 wk. | 8 wk. |
| Assay | | | 103.1 | 101.4 | 103.1 | 99.96 | 103.1 | 102.6 | 102.7 | 102.2 | 99.92 |
| Impurity A | 0.61 | 0.50% | BRL | 0.11 | 0.15 | 0.17 | BRL | 0.25 | 0.26 | 0.25 | 0.27 |
| Impurity B | 0.89 | 0.50% | BRL | BRL | BRL | BRL | BRL | BRL | BRL | BRL | BRL |
| Impurity C | 1.05 | 0.50% | BRL | 0.17 | 0.19 | 0.31 | BRL | 0.17 | 0.32 | 0.40 | 0.50 |
| Impurity D | 1.50 | 0.50% | BRL | BRL | BRL | BRL | BRL | BRL | BRL | BRL | BRL |
| Unk1 | 0.96 | 0.20% | 0.05 | 0.05 | BRL | BRL | 0.05 | BRL | BRL | BRL | BRL |
| Total Impurities | | 1.00% | 0.05 | 0.32 | 0.35 | 0.49 | 0.05 | 0.42 | 0.58 | 0.65 | 0.77 |

BRL; Below Report Level (0.05%)

TABLE 3
Stability of Formulation 1B

| | | | 40° C. | | | | 55° C. | | | | |
|---|---|---|---|---|---|---|---|---|---|---|---|
| | RRT | Limit | Time 0 | 2 wk. | 4 wk. | 8 wk. | Time 0 | 2 wk. | 4 wk. | 6 wk. | 8 wk. |
| Assay | | | 102.1 | 103.7 | 102.9 | 99.01 | 102.1 | 102.2 | 103.3 | 102.0 | 99.47 |
| Impurity A | 0.61 | 0.50% | BRL | 0.14 | 0.18 | 0.19 | BRL | 0.27 | 0.27 | 0.26 | 0.26 |
| Impurity B | 0.89 | 0.50% | BRL | BRL | BRL | BRL | BRL | BRL | BRL | BRL | BRL |
| Impurity C | 1.05 | 0.50% | BRL | 0.09 | 0.17 | 0.29 | BRL | 0.14 | 0.30 | 0.39 | 0.45 |
| Impurity D | 1.50 | 0.50% | BRL | BRL | BRL | BRL | BRL | BRL | BRL | BRL | BRL |
| Unk1 | 0.96 | 0.20% | 0.05 | 0.05 | BRL | BRL | 0.05 | BRL | BRL | BRL | BRL |
| Total Impurities | | 1.00% | 0.05 | 0.28 | 0.34 | 0.47 | 0.05 | 0.42 | 0.58 | 0.66 | 0.72 |

BRL; Below Report Level (0.05%)

TABLE 4
Stability of Formulation 1C

| | | | 40° C. | | | | 55° C. | | | | |
|---|---|---|---|---|---|---|---|---|---|---|---|
| | RRT | Limit | Time 0 | 2 wk. | 4 wk. | 8 wk. | Time 0 | 2 wk. | 4 wk. | 6 wk. | 8 wk. |
| Assay | | | 102.3 | 102.6 | 102.8 | 100.0 | 102.3 | 101.6 | 103.2 | 102.4 | 99.61 |
| Impurity A | 0.61 | 0.50% | BRL | 0.11 | 0.14 | 0.17 | BRL | 0.25 | 0.25 | 0.26 | 0.26 |
| Impurity B | 0.89 | 0.50% | BRL | BRL | BRL | BRL | BRL | BRL | BRL | BRL | BRL |
| Impurity C | 1.05 | 0.50% | BRL | 0.11 | 0.18 | 0.27 | BRL | 0.15 | 0.29 | 0.39 | 0.54 |
| Impurity D | 1.50 | 0.50% | BRL | BRL | BRL | BRL | BRL | BRL | BRL | BRL | BRL |
| Unk1 | 0.96 | 0.20% | 0.05 | 0.05 | BRL | BRL | 0.05 | BRL | BRL | BRL | BRL |
| Total Impurities | | 1.00% | 0.05 | 0.27 | 0.32 | 0.44 | 0.05 | 0.40 | 0.54 | 0.65 | 0.80 |

BRL; Below Report Level (0.05%)

TABLE 5
Stability of Formulation 1D

| | | | 40° C. | | | | 55° C. | | | | |
|---|---|---|---|---|---|---|---|---|---|---|---|
| | RRT | Limit | Time 0 | 2 wk. | 4 wk. | 8 wk. | Time 0 | 2 wk. | 4 wk. | 6 wk. | 8 wk. |
| Assay | | | 102.8 | 102.6 | 102.6 | 102.0 | 102.8 | 101.8 | 102.2 | 102.1 | 100.0 |
| Impurity A | 0.61 | 0.50% | BRL | 0.11 | 0.15 | 0.17 | BRL | 0.25 | 0.26 | 0.26 | 0.25 |
| Impurity B | 0.89 | 0.50% | BRL | BRL | BRL | BRL | BRL | BRL | BRL | BRL | BRL |
| Impurity C | 1.05 | 0.50% | BRL | 0.07 | 0.16 | 0.27 | BRL | 0.17 | 0.29 | 0.38 | 0.49 |
| Impurity D | 1.50 | 0.50% | BRL | BRL | BRL | BRL | BRL | BRL | BRL | BRL | BRL |
| Unk1 | 0.96 | 0.20% | 0.05 | 0.05 | BRL | BRL | 0.05 | BRL | BRL | BRL | BRL |
| Total Impurities | | 1.00% | 0.05 | 0.27 | 0.32 | 0.44 | 0.05 | 0.40 | 0.54 | 0.65 | 0.80 |

BRL; Below Report Level (0.05%)

TABLE 6
Stability of Formulation 1E

| | | | 40° C. | | | 55° C. | | | |
|---|---|---|---|---|---|---|---|---|---|
| | RRT | Limit | Time 0 | 2 wk. | 4 wk. | Time 0 | 2 wk. | 4 wk. | 6 wk. |
| Assay | | | 101.6 | 99.90 | 99.33 | 101.6 | 98.94 | 98.63 | 99.94 |
| Impurity A | 0.61 | 0.50% | BRL | 0.09 | 0.14 | BRL | 0.23 | 0.24 | 0.23 |
| Impurity B | 0.89 | 0.50% | BRL | BRL | BRL | BRL | BRL | BRL | BRL |
| Impurity C | 1.05 | 0.50% | BRL | 0.09 | 0.17 | BRL | 0.21 | 0.42 | 0.74 |
| Impurity D | 1.50 | 0.50% | BRL | BRL | BRL | BRL | BRL | BRL | BRL |
| Unk1 | 0.96 | 0.20% | 0.05 | 0.05 | BRL | 0.05 | BRL | BRL | BRL |
| Total Impurities | | 1.00% | 0.05 | 0.23 | 0.31 | 0.05 | 0.44 | 0.66 | 0.97 |

BRL; Below Report Level (0.05%)

TABLE 7
Stability of Formulation 1F

| | | | 40° C. | | | 55° C. | | | |
|---|---|---|---|---|---|---|---|---|---|
| | RRT | Limit | Time 0 | 2 wk. | 4 wk. | Time 0 | 2 wk. | 4 wk. | 6 wk. |
| Assay | | | 101.8 | 99.10 | 98.82 | 101.8 | 98.99 | 99.07 | 99.83 |
| Impurity A | 0.61 | 0.50% | BRL | 0.09 | 0.14 | BRL | 0.21 | 0.24 | 0.23 |
| Impurity B | 0.89 | 0.50% | BRL | BRL | BRL | BRL | BRL | BRL | BRL |
| Impurity C | 1.05 | 0.50% | BRL | 0.08 | 0.16 | BRL | 0.19 | 0.41 | 0.68 |
| Impurity D | 1.50 | 0.50% | BRL | BRL | BRL | BRL | BRL | BRL | BRL |
| Unk1 | 0.96 | 0.20% | 0.05 | 0.05 | 0.05 | 0.05 | BRL | BRL | BRL |
| Total Impurities | | 1.00% | 0.05 | 0.22 | 0.35 | 0.05 | 0.40 | 0.65 | 0.91 |

BRL; Below Report Level (0.05%)

TABLE 8
Stability of Formulation 1G

| | | | 40° C. | | | 55° C. | | | |
|---|---|---|---|---|---|---|---|---|---|
| | RRT | Limit | Time 0 | 2 wk. | 4 wk. | Time 0 | 2 wk. | 4 wk. | 6 wk. |
| Assay | | | 101.8 | 100.0 | 99.48 | 101.8 | 99.47 | 99.22 | 99.64 |
| Impurity A | 0.61 | 0.50% | BRL | 0.09 | 0.14 | BRL | 0.22 | 0.24 | 0.22 |
| Impurity B | 0.89 | 0.50% | BRL | BRL | BRL | BRL | BRL | BRL | BRL |
| Impurity C | 1.05 | 0.50% | BRL | 0.08 | 0.16 | BRL | 0.19 | 0.43 | 0.71 |
| Impurity D | 1.50 | 0.50% | BRL | BRL | BRL | BRL | BRL | BRL | BRL |
| Unk1 | 0.96 | 0.20% | 0.05 | 0.05 | BRL | 0.05 | BRL | BRL | BRL |
| Total Impurities | | 1.00% | 0.05 | 0.14 | 0.30 | 0.05 | 0.41 | 0.67 | 0.93 |

BRL; Below Report Level (0.05%)

As can be seen in Tables 2 to 8, the formulations maintained high concentrations of ketorolac as determined by chemical analysis. The formulations also had low levels of impurities following storage as determined by chemical analysis. “BRL” means that the impurity was below report level (0.05%). Relative retention time “RRT” is provided for each impurity. “Unknown” was abbreviated as “Unk” in the tables.

### Example 3

In order to determine the spray profiles, plume geometries, and particle sizes of Formulations 1D, 1H, 1I, and 1J, they were subjected to standardized droplet testing. A challenge of creating a ketorolac sublingual spray formulation is that it must be capable of producing spray droplets that are over 10 microns in diameter. Spray droplets 10 microns or smaller could be inhaled into the lungs. The optimal particle size for sublingual spray droplets is from 20 to about 200 microns in diameter. It is desirable for the formulation to have droplet sizes near 20 because this increases the surface area and increased surface area exposure is one factor that contributes to a high bioavailability. Sublingual formulations should be able to maintain a consistent droplet size throughout its shelf life.

Droplet analysis was conducted using standard laser analysis procedures known by those of skill in the art. Droplet size distribution (Dv10, Dv50, Dv90, and Span were tested at two distances, 3 cm and 6 cm). Dv10 refers to droplet size for which 10% of the total volume is obtained; Dv50 refers to droplet size for which 50% of the total volume is obtained; Dv90 refers to droplet size for which 90% of the total volume is obtained; Span refers to distribution span (Dv90−Dv10)/Dv50; % RSD refers to the percent relative standard deviation. Spray pattern, specifically Dmin, Dmax, and ovality ratio were tested at two distances, 3 cm and 6 cm. Dmin refers to the shortest diameter of the spray pattern in mm, Dmax refers to the widest diameter of the spray pattern in mm, and ovality ratio refers to the ratio of Dmax to Dmin. The spay pattern is measured after impact onto an appropriate target upon activation of a spray pump. The ovality ratio is useful as it provides information regarding the shape and density of the spray pump plume.

The results of these tests can be seen below in Tables 9 to 14. Applicant found during testing that formulations of the present invention yielded desirable droplet size distributions, spray patterns, and plume geometries for sublingual administration. The testing also revealed that the formulation dose remains consistent when administered with a spray pump.

TABLE 9
Spray Profile of Ketorolac Spray Formulation 1H, Droplet Size Distribution at 3 cm/6 cm

| | | 3 cm | | | | 6 cm | | | |
|---|---|---|---|---|---|---|---|---|---|
| Bottle | Actuation | Dᵥ10 | Dᵥ50 | Dᵥ90 | Span | Dᵥ10 | Dᵥ50 | Dᵥ90 | Span |
| 1 | 1 | 108.4 | 217.6 | 381.6 | 1.255 | 77.41 | 248.5 | 525.9 | 1.805 |
| | 2 | 68.49 | 159.8 | 362.8 | 1.842 | 93.37 | 249.2 | 505.8 | 1.655 |
| | 3 | 121.4 | 244.4 | 471 | 1.43 | 90.49 | 267.8 | 539.3 | 1.676 |
| 2 | 1 | 142.2 | 246.2 | 419.2 | 1.125 | 106.5 | 236.6 | 473.5 | 1.551 |
| | 2 | 144.9 | 258.4 | 459.4 | 1.217 | 116.4 | 251.7 | 495 | 1.504 |
| | 3 | 153.6 | 271.1 | 486.7 | 1.229 | 113.8 | 233.8 | 443.3 | 1.409 |
| 3 | 1 | 168.1 | 275.4 | 446.1 | 1.01 | 70.15 | 211.4 | 416.6 | 1.639 |
| | 2 | 164.6 | 312.1 | 582.7 | 1.34 | 62.32 | 194.9 | 417.2 | 1.821 |
| | 3 | 162.1 | 306.4 | 574.2 | 1.345 | 53.37 | 184.9 | 404.8 | 1.901 |

| | | | | | | | | |
|---|---|---|---|---|---|---|---|---|
| Average | 137.09 | 254.60 | 464.86 | 1.31 | 87.09 | 230.98 | 469.04 | 1.66 |
| Standard Deviation | 32.56 | 46.42 | 75.97 | 0.24 | 22.73 | 28.00 | 50.55 | 0.16 |
| % RSD | 23.75 | 18.23 | 16.34 | 17.94 | 26.09 | 12.12 | 10.78 | 9.63 |

TABLE 10
Spray Profile of Ketorolac Spray Formulation 1I, Droplet Size Distribution at 3 cm/6 cm

| | | 3 cm | | | | 6 cm | | | |
|---|---|---|---|---|---|---|---|---|---|
| Bottle | Actuation | Dᵥ10 | Dᵥ50 | Dᵥ90 | Span | Dᵥ10 | Dᵥ50 | Dᵥ90 | Span |
| 1 | 1 | 77.85 | 183.9 | 421.5 | 1.869 | 53.13 | 161.4 | 383.6 | 2.047 |
| | 2 | 82.28 | 195.9 | 450.9 | 1.882 | 56.84 | 170 | 396.2 | 1.996 |
| | 3 | 81.79 | 197.3 | 452.1 | 1.877 | 56.16 | 169.8 | 394.9 | 1.994 |
| 2 | 1 | 88.75 | 212.3 | 460.3 | 1.75 | 70.22 | 190.4 | 407.1 | 1.769 |
| | 2 | 85.68 | 200.2 | 433.9 | 1.739 | 70.24 | 185 | 384.3 | 1.697 |
| | 3 | 83.41 | 201.2 | 447.6 | 1.81 | 66.58 | 182.8 | 390.8 | 1.774 |
| 3 | 1 | 58.26 | 153.8 | 384.8 | 2.123 | 39.43 | 123.7 | 268.8 | 1.854 |
| | 2 | 60.27 | 156.9 | 371.9 | 1.986 | 39.17 | 122.8 | 272.8 | 1.903 |
| | 3 | 60.1 | 149.9 | 346.2 | 1.909 | 40.52 | 128.2 | 291.4 | 1.958 |

| | | | | | | | | |
|---|---|---|---|---|---|---|---|---|
| Average | 75.38 | 183.49 | 418.80 | 1.88 | 54.70 | 159.34 | 354.43 | 1.89 |
| Standard Deviation | 12.24 | 23.68 | 41.16 | 0.12 | 12.79 | 27.33 | 58.30 | 0.12 |
| % RSD | 16.24 | 12.90 | 9.83 | 6.31 | 23.38 | 17.15 | 16.45 | 6.44 |

TABLE 11
Spray Profile of Ketorolac Spray Formulation 1D, Unit Dose, at 3 cm

| | | | | | | Spray Pattern | | | Plume Geometry | |
|---|---|---|---|---|---|---|---|---|---|---|
| | Droplet Size Distribution | | | | | Dmin | Dmax | Ovality | Width | Angle |
| | Dᵥ10 | Dᵥ50 | Dᵥ90 | %<10 μ | Span | (mm) | (mm) | Ratio | (mm) | (°) |
| Actuation 1 | 12.04 | 27.99 | 109.5 | 5.82 | 3.483 | 22.3 | 33.5 | 1.503 | 35.3 | 59.3 |
| Actuation 2 | 13.39 | 27.65 | 143.5 | 2.721 | 4.706 | 20.9 | 25.7 | 1.233 | 28.8 | 50.6 |
| Actuation 3 | 14.47 | 35.67 | 246.9 | 3.488 | 6.516 | 21.1 | 30.0 | 1.427 | 19.6 | 35.0 |

TABLE 12
Spray Profile of Ketorolac Spray Formulation 1D, Multi-dose, at 3 cm

| | | | | | | Sptay Pattern | | | Plume Geometry | |
|---|---|---|---|---|---|---|---|---|---|---|
| | Droplet Size Distribution | | | | | Dmin | Dmax | Ovality | Width | Angle |
| | Dᵥ10 | Dᵥ50 | Dᵥ90 | %<10 μ | Span | (mm) | (mm) | Ratio | (mm) | (°) |
| Actuation 1 | 20.51 | 71.98 | 509.5 | 1.068 | 6.793 | 20.1 | 23.5 | 1.169 | 24.2 | 43.0 |
| Actuation 2 | 20.31 | 72.97 | 493.6 | 1.088 | 6.486 | 20.1 | 23.6 | 1.176 | 24.5 | 43.9 |
| Actuation 3 | 19.70 | 64.07 | 437.8 | 1.202 | 6.525 | 19.2 | 23.3 | 1.213 | 23.1 | 41.5 |

TABLE 13
Spray Profile of Ketorolac Spray Formulation 1D, Unit Dose, at 6 cm

| | | | | | | Spray Pattern | | | Plume Geometry | |
|---|---|---|---|---|---|---|---|---|---|---|
| | Droplet Size Distribution | | | | | Dmin | Dmax | Ovality | Width | Angle |
| | Dᵥ10 | Dᵥ50 | Dᵥ90 | %<10 μ | Span | (mm) | (mm) | Ratio | (mm) | (°) |
| Actuation 1 | 21.54 | 37.38 | 68.57 | 0.00 | 1.258 | 24.9 | 53.5 | 2.148 | 38.5 | 35.2 |
| Actuation 2 | 16.83 | 35.72 | 161.00 | 2.351 | 4.037 | 29.2 | 48.1 | 1.649 | 40.8 | 36.9 |
| Actuation 3 | 19.30 | 34.35 | 62.54 | 0.9693 | 1.259 | 29.6 | 52.1 | 1.757 | 35.9 | 32.6 |

TABLE 14
Spray Profile of Ketorolac Spray Formulation 1D, Multi-dose, at 6 cm

| | | | | | | Spray Pattern | | | Plume Geometry | |
|---|---|---|---|---|---|---|---|---|---|---|
| | Droplet Size Distribution | | | | | Dmin | Dmax | Ovality | Width | Angle |
| | Dᵥ10 | Dᵥ50 | Dᵥ90 | %<10 μ | Span | (mm) | (mm) | Ratio | (mm) | (°) |
| Actuation 1 | 21.56 | 49.57 | 508.6 | 1.185 | 9.823 | 24.4 | 40.5 | 1.664 | 37.0 | 34.2 |
| Actuation 2 | 21.44 | 44.35 | 352.3 | 1.603 | 7.461 | 25.3 | 40.5 | 1.601 | 37.0 | 34.2 |
| Actuation 3 | 21.38 | 45.77 | 412.5 | 1.511 | 8.546 | 24.5 | 41.9 | 1.710 | 41.3 | 45.5 |

We claim:

1. A room temperature storage stable sublingual spray formulation comprising:
a. from about 0.1% w/w to about 35% w/w ketorolac or a salt thereof;
b. from about 0.1% w/w to about 95% w/w water;
c. from about 0.001% w/w to about 1% w/w of a buffer salt; and
d. from about 0.001% w/w to about 1% w/w antioxidant,
wherein the pH of the formulation is from about 5 to about 9.

2. The formulation of claim 1 wherein the ketorolac salt is ketorolac tromethamine.

3. The formulation of claim 1 wherein the buffer salt is selected from the group consisting of a sodium, potassium, or calcium salt of citric acid, acetic acid, phosphoric acid, boric acid malic acid, adipic acid, fumaric acid, tartaric acid, palmitic acid, and a combination thereof.

4. The formulation of claim 1 further comprising a solvent selected from the group consisting of ethyl alcohol, propylene glycol, polyethylene glycol, glycerol and a combination thereof.

5. The formulation of claim 1 wherein the buffer salt is sodium citrate.

6. The formulation of claim 1 wherein the pH is from about 6 to about 8.

7. The formulation of claim 1 wherein the antioxidant is selected from the group consisting of ascorbyl palmitate, ascorbic acid, sodium ascorbate, alpha tocopherol, butylated hydroxytoluene, butylated hydroxyanisole, cysteine HCl, citric acid, ethylene diamine tetra acetic acid (EDTA), methionine, sodium metabisulfite, sodium bisulfate, propyl gallate, thioglycerol, and combinations thereof.

8. The formulation of claim 1 wherein the antioxidant is sodium ascorbate.

9. The formulation of claim 1 that is capable of producing a droplet size distribution wherein the mean Dv(10) is from about 10 to about 170 microns during administration.

10. The formulation of claim 1 that is capable of producing a droplet size distribution wherein the mean Dv(50) is from about 20 to about 315 microns during administration.

11. The formulation of claim 1 that is capable of producing a droplet size distribution wherein the mean Dv(90) is from about 60 to about 585 microns during administration.

12. The formulation of claim 1 that is capable of producing a spray pattern wherein the Dmin is from about 15 to about 35 millimeter during administration.

13. The formulation of claim 1 that is capable of producing a spray pattern wherein the Dmax is from about 20 to about 55 millimeter during administration.

14. The formulation of claim 1 that is capable of producing a spray pattern wherein the ovality ratio is about 1 to 2.5 during administration.

15. The formulation of claim 1 that is capable of producing a plume geometry wherein the width is about 15 to 45 millimeter during administration.

16. The formulation of claim 1 that is capable of producing a plume geometry wherein the angle is about 30 to 65 degrees.

17. A room temperature storage stable, sublingual spray formulation comprising:
a. from about 10% w/w to about 20% w/w ketorolac salt;
b. from about 65% w/w to about 85% w/w water;
c. from about 0.001% w/w to about 1% w/w of a buffer salt selected from the group consisting of a sodium salt of citric acid, phosphoric acid and a combination thereof; and
d. from about 0.001% w/w to about 1% w/w antioxidant,
wherein the pH of the formulation is from about 5 to about 9.

18. A method of treating pain comprising administering the formulation of claim 1 to a patient in need thereof.

19. The method of claim 18 wherein the spray pumps deliver about 50 to about 200 μL of the formulation of claim 1 under the tongue.

20. The method of claim 18 wherein the pain is caused by a migraine headache.

21. The method of claim 18 wherein the pain is the result of surgery.
